(19) United States
(12) Patent Application Publication
(10) Pub. No.: US 2026/0196332 A1
(43) Pub. Date: Jul. 9, 2026

(54) METHOD OF TREATING NEURODEGENERATIVE BRAIN DISEASE WITH VR STIMULATION THAT IS MAPPED TO PATIENT-SPECIFIC PARAMETERS

(71) Applicant: Clarity Technologies SASU, Paris (FR)

(72) Inventors: Carolina Reis, Paros (FR); Raphael Certain, Paris (FR)

(21) Appl. No.: 19/445,371

(22) Filed: Jan. 9, 2026

Related U.S. Application Data
(60) Provisional application No. 63/743,567, filed on Jan. 9, 2025.

(51) Int. Cl. G16H 20/70

(52) U.S. Cl. CPC G16H 20/70

(57) ABSTRACT

Disclosed herein are systems and methods for personalizing neuromodulation therapy delivered within virtual reality environments based on patient-specific information indicative of a clinical or functional status of a user. Such information may include clinical, symptomatic, behavioral, and performance-derived data, as well as information obtained from external systems or indicative of therapy delivery, adherence, or engagement. The system integrates structured external data—such as clinical assessments, caregiver reports, and medical records—with task performance metrics and contextual physiological signals collected during immersive virtual-reality sessions. A performance module administers standardized or adaptive tasks within virtual environments, while a scoring module evaluates multimodal inputs to determine the relative significance of functional deficits across cognitive, motor, affective, or behavioral domains. A mapping module uses these weighted evaluations to generate and adjust individualized therapeutic protocols by selecting and parameterizing virtual environments, task structures, and neuromodulation methods, including sensory stimulation delivered at specific frequencies.

### CROSS-REFERENCE TO RELATED APPLICATION

This application claims the priority benefit of U.S. provisional patent application No. 63/743,567 filed Jan. 9, 2025, which are incorporated by reference herein in their entirety.

### FIELD OF THE DISCLOSURE

The present disclosure relates to systems and methods for mapping patient-specific information indicative of clinical or functional status to virtual-reality-based neuromodulation protocols for personalized therapeutic intervention.

### BACKGROUND

Neurological, neurodegenerative, and neuropsychiatric disorders represent a major and growing global health burden. Many such conditions are characterized by progressive impairment of cognitive, motor, emotional, or autonomic function and are associated with significant reductions in quality of life. Despite advances in diagnosis and clinical management, a large number of these disorders lack effective disease-modifying treatments, and therapeutic strategies are often limited to symptomatic relief rather than addressing underlying pathological mechanisms. Moreover, disease progression, symptom manifestation, and treatment response may vary substantially across individuals and over time, complicating the development of universally effective therapeutic interventions.

Neuromodulation has emerged as a promising therapeutic approach for influencing neural activity in order to alleviate symptoms, manage disease progression, or modulate dysfunctional biological processes associated with neurological and neuropsychiatric disorders. Neuromodulation techniques may include electrical stimulation, sensory stimulation, peripheral nerve stimulation, or other forms of externally applied stimulation delivered to influence neural activity and its downstream effects. Such stimulation may be delivered using a variety of temporal patterns or frequencies, which can differentially shape neural dynamics and associated biological responses.

Importantly, the effects of neuromodulation may extend beyond immediate modulation of neural firing or circuit dynamics and may influence downstream molecular, cellular, or systemic processes, including inflammatory signaling, protein aggregation pathways, vascular or metabolic function, synaptic remodeling, and neuroglial interactions. By way of example, gamma-frequency sensory stimulation has been shown in certain contexts to entrain neural oscillations and has been associated with downstream biological effects such as altered protein aggregation, modulation of neuroinflammatory pathways, and changes in network-level synchrony.

However, many existing neuromodulation approaches rely on fixed or preconfigured stimulation protocols that do not adequately account for inter-subject variability, intra-subject variability, or longitudinal disease progression. Therapeutic parameters are often selected based on population-level assumptions or static clinical categorizations, rather than being dynamically aligned to an individual patient's evolving functional status, symptom profile, or therapeutic response. As a result, neuromodulation therapies may exhibit variable efficacy, limited durability, or reduced clinical relevance across different patients and stages of disease.

At the same time, the availability of patient-specific information capable of informing therapeutic decisions—including clinical assessments, behavioral measures, performance data, and physiological or biomarker-related indicators—is increasing. There remains a need for systems and methods that can leverage such information to personalize neuromodulation therapy and adapt therapeutic protocols over time in a manner that reflects individual functional needs and disease trajectories.

### SUMMARY OF THE DISCLOSURE

The present disclosure relates to systems and methods for delivering personalized neuromodulation therapy within immersive or semi-immersive virtual-reality environments based on patient-specific information indicative of a clinical or functional status of a patient. Such patient-specific information may include clinical, symptomatic, behavioral, and performance-derived information, as well as information derived from sensors and biological indicators and in some embodiments may further include information indicative of task delivery, adherence, or engagement with therapy The disclosed system integrates structured external clinical inputs—such as clinical and physiological assessments, caregiver observations, and reported symptoms—with task performance data and sensor-derived behavioral measures acquired during interaction with virtual environments.

In accordance with one or more embodiments, a performance module administers standardized or adaptive tasks within virtual-reality environments to elicit observable behavioral, cognitive, motor, or affective responses. A scoring module evaluates these responses, alone or in combination with external clinical information, to generate weighted representations of functional domains relevant to therapy personalization. A mapping module uses the weighted representations to assemble and parameterize individualized therapeutic protocols by selecting virtual environments, task logic structures, and neuromodulation methods, including sensory stimulation delivered through visual, auditory, or other modalities.

The disclosed system supports delivery of neuromodulation therapy in supervised, semi-supervised, or unsupervised settings, including home-based use, while maintaining execution-fidelity monitoring and optional remote oversight. In accordance with one or more embodiments, therapeutic protocols may be assembled and parameterized on a patient-specific basis using information indicative of the patient's clinical or functional status, rather than being fixed or predefined. In some embodiments, such protocols may be further updated longitudinally based on newly acquired patient-specific information obtained during therapy or received from external sources, enabling closed-loop personalization driven by functional outcomes over time. Therapeutic updates may be implemented automatically or subject to clinician review and authorization. By embedding neuromodulation within task-based virtual-reality contexts aligned with patient-specific functional impairments, the disclosed systems and methods enable context-dependent therapy that adapts to clinical presentation and performance over time, thereby improving precision, scalability, and clinical relevance in the treatment of neurological and neurodegenerative disorders.

### DESCRIPTIONS OF THE DRAWINGS

FIG. 1: illustrates a method of mapping patient-specific parameters to virtual reality environments and neuromodulation protocols to treat neurodegenerative or brain disorders, according to an embodiment.

FIG. 2: illustrates a Performance Module, according to an embodiment.

FIG. 3: illustrates a Scoring Module, according to an embodiment.

FIG. 4: illustrates a Mapping Module, according to an embodiment.

FIG. 5: illustrates a Data Structuring and Exchange Module, according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

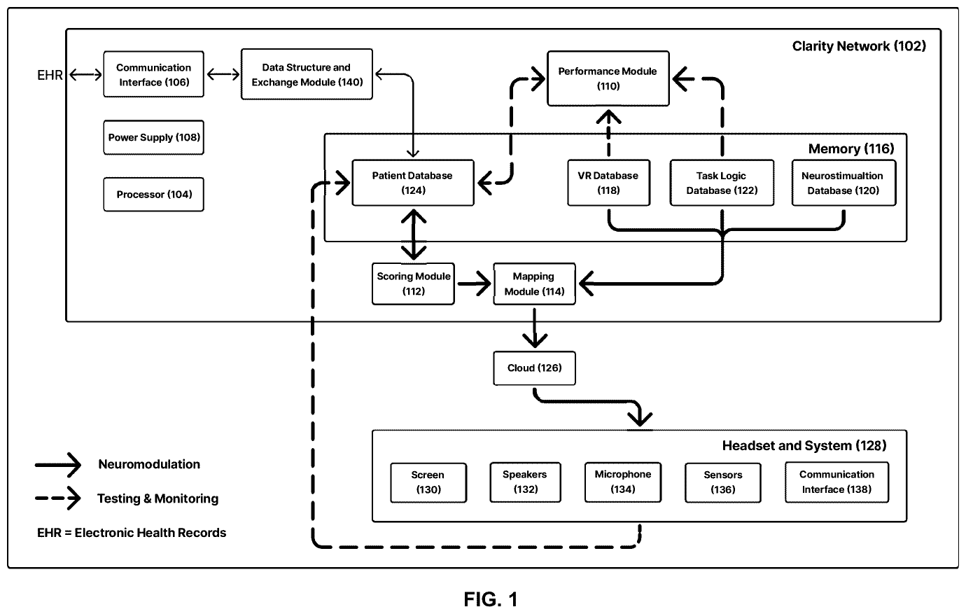

FIG. 1 illustrates a method of mapping patient-specific clinical, behavioral, symptomatic, and performance-related parameters to virtual reality environments and neuromodulation protocols to treat neurodegenerative or brain disorders. The figure illustrates an embodiment of a system architecture 102 (“Clarity Network”) configured to deliver personalized neuromodulation and virtual-reality-based therapeutic protocols by integrating clinical data, performance data, environmental context, and multimodal physiological measurements. As shown, the system may include one or more modules, databases, and communication interfaces that cooperate to administer testing, monitoring, and treatment sessions through a headset and system 128, while continuously collecting data for personalization, safety assurance, and remote clinical oversight.

In the context of the present disclosure, therapeutic tasks may comprise structured activities configured to engage one or more functional domains of a patient during a neuromodulation session. Such therapeutic tasks may include, but are not necessarily limited to, cognitive tasks, motor tasks, affective or emotional processing tasks, sensory-perceptual tasks, or combined functional activities. Therapeutic tasks may involve mental actions, physical actions, or coordinated interactions within a virtual-reality environment, and may be configured with adjustable parameters such as duration, difficulty, pacing, interaction modality, or stimulus characteristics.

In the illustrated embodiment, external information from electronic health-record (EHR) systems or connected devices may be exchanged with the Clarity Network 102 through a communication interface 106 and processed by a data structuring and exchange module 140. The processed information is stored in a patient database 124 and made available to internal components including a scoring module 112, a mapping module 114, and a performance module 110. The system may further include a memory 116 comprising a virtual reality database 118, a task logic database 122, and a neurostimulation database 120, each of which supplies environment assets, task rules, and stimulation parameters, respectively. These components support dynamic generation of testing and therapeutic sessions that are delivered to the user through the headset and system 128, which includes displays, speakers, electrodes, sensors, and communication interfaces 138 for data capture and bidirectional communication. As illustrated, the system may operate in open-loop or closed-loop configurations, with data flowing from the headset and system 128 back to the Clarity Network 102 to support longitudinal monitoring, adaptive therapy, and optional external oversight or review. The clarity network 102 may support high-speed processing, reliable data exchange, and scalability, ensuring efficient handling of both real-time patient interactions and long-term therapeutic data management.

Further, embodiments may include a processor 104, also referred to as a central processing unit (CPU), which may facilitate the operation of the system by executing instructions stored in the memory 116. In some embodiments, the processor 104 may additionally comprise a graphics processing unit (GPU), system-on-chip (SoC), or other processing circuitry suitable for handling computational tasks associated with the Clarity Network 102. The processor 104 may include suitable logic, circuitry, interfaces, and/or code configured to fetch, decode, and execute instructions, perform arithmetic and logical operations, and manage the flow of data among the modules and databases shown in FIG. 1.

The processor 104 may include components such as arithmetic and logic units, control units, caches, registers, and other subsystems that provide fast access to frequently used instructions and data. These components may be designed to minimize the latency associated with manipulating data and carrying out operations required by the performance module 110, scoring module 112, mapping module 114, and the data structuring and exchange module 140. In some embodiments, the instruction set architecture (ISA) of the processor 104 may define the instruction formats, addressing modes, and exception-handling mechanisms used to support execution of the system's software components.

In certain embodiments, processing responsibilities may be distributed between the processor 104, local processing hardware within the headset and system 128, and remote computing resources such as cloud services 126. This distributed architecture may allow the Clarity Network 102 to support low-latency rendering, real-time coordination of therapeutic stimulation, adaptive feedback processing, and secure long-term data storage while maintaining efficient communication with external systems through the communication interfaces 106 and 138. Together, these processors and processing subsystems enable coordinated operation of the memory 116, patient database 124, and other modules to deliver personalized testing, monitoring, and therapeutic protocols.

Further, embodiments may include a communication interface 106, which may be a hardware or software component that enables communication between two or more electronic devices or systems. The communication interface 106 may include a set of protocols, rules, and standards that define how information is transmitted and received between devices. The communication interface 106 may be implemented using a physical connector, wireless network, or software application, and may include components such as drivers, software libraries, and firmware that control and manage the communication process. In some embodiments, the communication interface 106 may be compatible with USB, Bluetooth, Wi-Fi, or other wired or wireless technologies.

The communication interface 106 may communicate with a network. Examples of networks may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN). In some embodiments, the communication interface 106 may facilitate data exchange between the Clarity Network 102 and external systems, such as clinical servers providing clinician-entered or assessment-related information, cloud-based computing resources 126, or connected applications used by caregivers or patients for reporting symptoms, performance, or session-related information. In some embodiments, data received through the communication interface 106 may be routed to the data structuring and exchange module 140 for validation and formatting before being written to the patient database 124.

Further, embodiments may include a power supply 108, which may be an electrical device or system used to convert electrical power from a source to a specific form or voltage that the electronic components of the system can utilize. The power supply 108 may be designed to regulate and control output power to ensure that the device or system receives the correct amount of electrical energy without damage. The power supply 108 may include components such as transformers, rectifiers, filters, voltage regulators, and control circuits that work together to provide the desired output voltage and current. The input power source may be an AC or DC supply, such as a battery, wall outlet, or generator.

The power supply 108 may be classified according to parameters such as output voltage type, power rating, efficiency, regulation, or intended application. In some embodiments, the power supply 108 may include protection mechanisms such as overvoltage protection, overcurrent protection, short-circuit protection, and thermal protection to ensure safe and reliable operation. In certain embodiments, the power supply 108 may distribute electrical power to the processor 104, the communication interfaces 106 and 138, the memory 116, and associated modules, supporting operation of the Clarity Network 102 and the headset and system 128. The power supply 108 may also cooperate with onboard or external charging systems to maintain continuous operation during extended or repeated testing, monitoring, or therapeutic sessions, including in home-based or unsupervised deployment scenarios.

Further, embodiments may include a performance module 110, which may be configured to administer, manage, and adapt testing procedures within the virtual environment based on clinical indications and patient-specific data. The performance module 110 may receive as input a clinical indication or therapeutic goal that defines the domains to be assessed or monitored, such as cognitive, motor, affective, or neural functions. Based on this information, the performance module 110 may generate or select a schedule of testing sessions, retrieving virtual environments from the virtual reality database 118 and associated prompts from the task logic database 122 to configure structured, task-specific scenarios. The performance module 110 may define the overall testing session structure, while delegating the presentation and interaction flow of each individual task to the task logic database 122. Each testing session may be designed to evaluate one or more features relevant to the patient's condition, including attention, memory, reaction time, coordination, emotional reactivity, among others. In some embodiments, the performance module 110 may define execution parameters such as the number of trials, repetitions, difficulty progression, and performance thresholds required to evaluate user capability or detect functional deficits.

In some embodiments, the performance module 110 may dynamically adapt the testing configuration using data from multiple sources, including external inputs from caregivers or medical teams, wearable sensors, prior performance results, or treatment outcomes collected during therapy sessions. These data streams enable the performance module 110 to refine task type, duration, and difficulty so that each testing cycle remains clinically relevant and personalized to the patient's evolving condition. During operation, the performance module 110 may coordinate the delivery of the configured environments and prompts to the headset and system 128, while recording behavioral and physiological responses through sensors 136 and microphone 134. The resulting data may be stored in the memory 116 and written to the patient database 124, where they are analyzed by the scoring module 112 and may be used by the mapping module 114 to support longitudinal monitoring and closed-loop adjustment of therapeutic protocols. In some embodiments, summarized performance data may also be processed by the data structuring and exchange module 140 and transmitted through the communication interface 106 to authorized clinical teams for review or ongoing therapy management, and in some embodiments to caregivers or patients.

Further, embodiments may include a scoring module 112, which may be configured to evaluate, weight, and prioritize patient-specific data to determine their clinical significance for therapy adaptation. The scoring module 112 may analyze multimodal data entries originating from the performance module 110, including behavioral and physiological responses obtained during testing or therapeutic sessions, and may further include indicators of task delivery, adherence, or engagement, as well as external information received through the data structuring and exchange module 140, such as clinical reports, caregiver observations, or wearable sensor outputs. The scoring module 112 may apply predefined algorithms, rule-based logic, or machine-learning models to quantify the relevance and reliability of each data entry relative to the patient's clinical presentation, symptom profile, functional impairments, or comorbid conditions. In some embodiments, the scoring rules comprise any combination of heuristic thresholds, statistical inference, and learned models, and may be updated or replaced without changing the overall personalization architecture described herein.

In some embodiments, the scoring module 112 may generate weighted scores or confidence values describing the magnitude and clinical importance of observed changes or deficits across functional domains such as cognition, affect or motor coordination. The scoring module 112 may establish hierarchical thresholds—e.g., low, medium, or high significance—to identify which features should be incorporated into the subsequent mapping process. For example, concordant findings across different sources (such as impaired spatial-navigation performance during a VR task and corresponding deficits identified in external standardized cognitive assessments) may receive a higher significance score than isolated or inconsistent findings.

The scoring outputs may be transmitted directly to the mapping module 114, which uses these results to adjust therapeutic parameters, virtual-reality task configurations, or neuromodulation protocols between sessions and/or during a session based on updated scoring outputs or session-level performance indicators.. In some embodiments, the scoring module 112 may store evaluated results and scoring rationale in the patient database 124 to maintain a longitudinal scoring history, support population-level model refinement, or enable clinician review and remote patient monitoring through the data structuring and exchange module 140. In certain embodiments, the scoring module 112 may update its evaluation parameters iteratively as new data become available, allowing continuous refinement of predictive accuracy and personalization of therapeutic protocols within the Clarity Network 102.

Further, embodiments may include a mapping module 114, which may be configured to generate, adapt, and schedule therapeutic protocols based on the significance-weighted results produced by the scoring module 112. The mapping module 114 may receive as input the prioritized scores, confidence values, or threshold classifications that indicate the functional, cognitive, motor or affective domains in which the patient demonstrates impairment or meaningful clinical change. Based on these results, the mapping module 114 may select one or more therapeutic targets—such as memory, visuospatial processing, attention, language, executive function, emotional regulation, or motor coordination—and determine the corresponding stimulation modalities, task types, and virtual-environment configurations appropriate for treatment.

In some embodiments, the mapping module 114 may retrieve parameter sets from the neurostimulation database 120 and associate them with virtual-reality environments from the virtual reality database 118 and task structures from the task logic database 122. These associations may define how sensory stimulation, neuromodulation methods, or task-based intefnotractions are combined during therapy. The mapping module 114 may also determine execution parameters for each therapeutic session, including stimulation modality, frequency, intensity, timing, session duration, and repetition schedule. In certain embodiments, the mapping module 114 may assign higher therapeutic weight to domains with stronger or more consistent deficits based on weighted representations generated by the scoring module 112 the scoring module 112, or may prioritize tasks and stimulation paradigms known to engage the clinically implicated functional domains. In some embodiments, the mapping module 114 may further adjust the therapeutic protocol over time as the patient's profile evolves, decreasing emphasis on domains that demonstrate improvement or plateauing performance and reallocating session time, stimulation parameters, or task types toward domains that newly emerge as clinically significant.

The generated therapy configuration may be transmitted to the headset and system 128, where the selected virtual environments, task rules, and stimulation parameters are delivered during therapy sessions. In some embodiments, the mapping module 114 may update the therapeutic protocol between sessions or in real time based on weighted representations generated by the scoring module 112, optionally informed by supporting physiological or neural signals recorded by sensors 136 and microphone 134 as contextual indicators of task execution or symptom expression, or by external inputs processed by the data structuring and exchange module 140. Such adaptive processes may support iterative optimization of therapy schedules and neuromodulation parameters. The mapping module 114 may further store the resulting protocol selections in the patient database 124 to maintain a longitudinal therapeutic record and support subsequent iterations of the personalization cycle within the Clarity Network 102.

Further, embodiments may include a memory 116, which may store data collected or generated within the Clarity Network 102, including performance data and task-derived metrics, sensor measurements captured during interaction, intermediate analysis results, stimulation parameters, and virtual-environment configuration data. In some embodiments, the memory 116 may include suitable logic, circuitry, and/or interfaces configured to store machine code and computer programs comprising code sections executable by the processor 104. The memory 116 may additionally store instructions, datasets, or temporary working data used by modules of the system during operation. Examples of implementations of the memory 116 may include, but are not limited to, Random Access Memory (RAM), Read-Only Memory (ROM), flash memory, a Hard Disk Drive (HDD), and/or a Secure Digital (SD) card.

Further, embodiments may include a virtual reality database 118, which may store, manage, and deliver immersive visual and auditory environments for therapeutic and assessment applications. The virtual reality database 118 may comprise preconfigured and customizable multimedia content that forms the sensory backdrop for cognitive, emotional, and motor interventions. In some embodiments, the virtual reality database 118 may contain 3D scenes, textures, animations, ambient audio, and environmental soundscapes that can be rendered in real time through the headset and system 128 to create fully immersive experiences. Example environments may include calming natural landscapes, daily-living simulations, gamified settings, or personalized scenes composed of familiar photographs or locations designed to increase relevance and therapeutic salience for the patient.

In some embodiments, the virtual reality database 118 may interface with the task logic database 122, which defines task rules, feedback mechanics, and difficulty progression within these environments, allowing the same multimedia assets to be used flexibly across testing and therapeutic contexts. The virtual reality database 118 may further interoperate with the neurostimulation database 120, which defines the modulation frequency, intensity, or phase parameters for any visual or auditory stimulation embedded within the virtual content. For instance, while the virtual reality database 118 provides the rendered objects or animations, the neurostimulation database 120 governs how their brightness or timing characteristics are modulated to achieve specific entrainment frequencies to impact specific behaviour.

In some embodiments, the virtual reality database 118 may support dynamic personalization, enabling users or caregivers to upload custom images, sounds, or videos that are automatically formatted and integrated into the immersive environment. The virtual reality database 118 may also contain collections of themed scenarios, such as meditation, relaxation, or guided-breathing or mindfulness environments, that may be selected and adapted by the mapping module 114 based on patient-specific therapeutic goals. Each environment stored in the virtual reality database 118 may be optimized for low-latency rendering, spatial-audio alignment, and seamless integration with sensors 136 within the headset and system 128, ensuring high fidelity and real-time responsiveness during therapy delivery.

Further, embodiments may include a neurostimulation database 120, which may store and organize detailed protocols for delivering targeted stimulation tailored to preserving, improving or slowing down specific cognitive, affective, motor, or neural impairments. The neurostimulation database 120 may serve as a library of neuromodulation methods comprising visual, auditory, tactile, and vibrotactile stimulation, as well as neurofeedback and electrical stimulation methods such as transcranial alternating current stimulation (tACS) and transcranial direct current stimulation (tDCS). The neurostimulation database 120 may also reference stimulation protocols for peripheral and deep brain targets, including vagus nerve stimulation, ultrasound neuromodulation, deep brain stimulation (DBS), or pharmacological agents with neuromodulatory effects.

Each method stored in the neurostimulation database 120 may be associated with a corresponding set of stimulation parameters, including frequency, amplitude, phase, waveform shape, carrier type, spatial pattern, electrode configuration, duty cycle, and duration per activation event. For example, visual stimulation protocols may specify flicker frequency, contrast modulation, luminance envelopes, or phase alignment with EEG activity. Auditory stimulation protocols may include amplitude-modulated tones, rhythmic click-trains, or broadband transients delivered at therapeutic frequencies. Tactile or mechanical protocols may define haptic pulse sequences or vibration envelopes, while electrical stimulation protocols may define electrode placement, current density, or polarity for a given neural or peripheral target.

The neurostimulation database 120 provides stimulation parameters to the screen 130, speakers 132, electrodes as part of sensors 136, and haptic actuators within the headset and system 128. In some embodiments, the neurostimulation database 120 may contain rules or parameter ranges that support dynamic adaptation based on real-time behavioural feedback.

In some embodiments, the neurostimulation database 120 may contain rules or parameter ranges that support adaptive selection of stimulation parameters based on changes in patient-specific clinical, symptomatic, or performance-derived information observed across therapy sessions. For example, longitudinal task outcomes administered through the performance module 110 may indicate worsening memory recall, slowed response initiation, increased motor variability, or changes in affective reactivity consistent with progression or fluctuation of a clinical symptom profile. In response, the mapping module 114 may adjust stimulation parameters such as frequency, modality, session structure, or therapeutic emphasis to target the functional domains exhibiting the greatest deviation from baseline or expected response.

In some embodiments, the neurostimulation database 120 may store calibration profiles defining stimulation parameter sets associated with differential therapeutic effects across symptom domains. For example, during an initial assessment or early treatment phase, the system may administer therapy segments using different stimulation configurations while monitoring task-based performance outcomes or symptom-report measures collected during or after sessions. Based on observed differences in cognitive performance, behavioral regulation, or symptom expression, the mapping module 114 may select or refine stimulation parameters for subsequent therapy cycles. Such calibration profiles may be updated longitudinally as changes in clinical presentation or functional capacity are detected over time.

Through this design, the neurostimulation database 120 functions as the centralized repository of validated stimulation parameters and delivery constraints, while the mapping module 114 selects, sequences, and applies these parameters within the appropriate virtual-environment and task context to generate individualized therapeutic protocol.

In the context of the present disclosure, therapeutic tasks may comprise structured activities configured to engage one or more functional domains of a patient during a neuromodulation session. Such therapeutic tasks may include, but are not necessarily limited to, cognitive tasks, motor tasks, affective or emotional processing tasks, sensory-perceptual tasks, or combined functional activities. Therapeutic tasks may involve mental actions, physical actions, or coordinated interactions within a virtual-reality environment, and may be configured with adjustable parameters such as duration, difficulty, pacing, interaction modality, or stimulus characteristics, including but not limited to intensity, frequency, timing, duty cycle, spatial distribution, or modality-specific presentation features.

Sensory stimulation, as used herein, may include the delivery of temporally patterned stimuli such as oscillations, pulses, pulse trains, flashes, or modulated signals. Sensory stimulation may target one or more frequency ranges and may be delivered using different modalities, including visual, auditory, mechanical, electrical, tactile, magnetic, or combinations thereof. In some embodiments, multiple sensory stimulation modalities may be coordinated such that their temporal patterns are synchronized, phase-aligned, offset, or ordered relative to one another in a predefined or adaptive manner. For example, a visual stimulus may be delivered at a first frequency while an auditory stimulus is delivered at a second frequency, either concurrently or sequentially. In other embodiments, sensory stimulation modalities may be alternated rather than presented simultaneously. Sensory stimulation may be delivered alone or in combination with other neuromodulation techniques, including but not limited to transcranial electrical stimulation, transcranial magnetic stimulation, ultrasound neuromodulation, or peripheral nerve stimulation. Stimulation may be delivered continuously, intermittently, or with purposeful interruptions, including to reduce habituation, maintain engagement, or support therapeutic objectives.

In some embodiments, the neurostimulation database 120 may store schemas for multimodal stimulation designed to produce synergistic effects across effects yielded by sensory and electrical neuromodulation methods. For instance, the database may define combined transcranial alternating current stimulation (tACS) and visual-flicker protocols in which the phase of the applied electrical field is aligned with the phase of a visual stimulus.

In one clinical example, a therapeutic schema may be configured to address deficits in episodic memory. The mapping module 114 may select a protocol in which transcranial alternating current stimulation (tACS) is applied over frontal cortical regions functionally connected to medial temporal lobe (MTL) or default mode network (DMN) circuitry, while concurrent visual flicker is delivered within a virtual-reality environment configured to probe spatial-navigation behaviors in order to entrain the hippocampus, at a temporally coordinated frequency. Such combined stimulation may be intended to improve memory-related performance measures by promoting coordinated activation of neural circuits implicated in episodic memory encoding and retrieval. Selection and adjustment of the multimodal stimulation parameters may be informed by patient-specific clinical presentation, symptom reports, and performance-derived measures obtained during memory-related tasks administered by the performance module 110. For example, changes in recall accuracy, response latency, or error patterns across sessions may be used by the scoring module 112 and mapping module 114 to refine stimulation frequency, cortical targeting, session duration, or relative weighting of sensory versus electrical stimulation components over time.

In some embodiments, therapeutic protocols may be configured to operate alongside other clinical interventions administered independently of the system.

In one embodiment, the system may coordinate sensory or neuromodulatory stimulation with adjunct mechanical stimulation, such as rhythmic actuation of facial or cervical tissues delivered through external accessories. Such mechanical patterns may be intended to support physiological processes—including cerebrospinal-fluid circulation, lymphatic clearance, or vascular dynamics—that operate independently of direct neural entrainment. When used, these mechanical-stimulation patterns may be temporally aligned with visual or auditory stimulation to maintain coherence across therapeutic modalities.

In other embodiments, the system may support pharmacological combination therapies, in which sensory stimulation is administered alongside medications prescribed as part of the patient's standard clinical care. The system may adjust task difficulty, stimulation timing, or scheduling logic based on medication timing, recorded symptomatic fluctuations, or clinician-entered treatment plans.

These combination-therapy options may be activated only when appropriate for a given indication, enabling the system to integrate complementary physiological or pharmacological interventions while keeping neuromodulation delivery strictly governed by the neurostimulation database 120 and mapping module 114.

Further, embodiments may include a task logic database 122, which serves as a repository of task structures, interaction rules, and prompting configurations used to implement cognitive, motor or affective tasks. The task logic database 122 may store predefined logic templates specifying how prompts, cues, or stimuli are presented—such as written or spoken instructions, visual indicators directing gaze, auditory tones signaling timing, or haptic pulses—and how user responses (e.g., verbal replies, gaze shifts, gestures, or motor actions) are to be recorded or evaluated through the corresponding sensors or input channels. These stored templates may be referenced and executed by other system components, including the performance module 110 during testing and monitoring phases or the mapping module 114 during therapeutic stimulation delivery.

The task logic database 122 may contain predefined rule sets describing task timing, sequencing, repetition, difficulty progression, and modality selection. In some embodiments, the database may also store metadata defining the interaction parameters required for each task, such as the number of stimuli, response format, or required sensor inputs (e.g., eye-tracking or speech data).

The task structures stored within the task logic database 122 may be derived from standardized clinical assessments or validated cognitive and motor paradigms. For example, a word-recall task used in a clinical setting may be represented in the database as a structured digital template specifying presentation order, recall timing, and response mode (verbal, visual, or motor). The database may further include references to corresponding stimulation parameters stored within the neurostimulation database 120, enabling synchronized sensory stimulation during task execution.

The task logic database 122 may include reference associations between task types and the physiological, cognitive, affective, or motor domains they are designed to engage. These associations do not perform the mapping between patient data and domain deficits but instead serve as metadata accessible to the mapping module 114. When the mapping module 114 identifies a specific domain to target—such as free recall, inhibitory control, or spatial navigation—it may use these stored associations to retrieve the corresponding task definitions, rules, and materials from the task logic database 122 for integration with the virtual environments and stimulation parameters defined in the virtual reality database 118 and neurostimulation database 120.

In some embodiments, the task logic database 122 may store event-driven or conditional rule definitions that describe how tasks may adapt under specific conditions, such as varying difficulty or timing based on behavioral performance or physiological input. These definitions represent the conditions and rule parameters but are not executed by the database itself; instead, the adaptive logic is carried out by the performance module 110 or mapping module 114 when they retrieve and implement the stored configurations.

The task logic database 122 may also store associations with assets from the virtual reality database 118, such as links to specific environments, object libraries, or multimedia elements, enabling efficient retrieval and presentation of content during task execution. Through these stored relationships, the database provides the underlying framework for configuring immersive cognitive and therapeutic experiences that may be tailored to the patient's clinical profile.

In operation, the task logic database 122 serves as a structured repository of task definitions and associated rules that may be retrieved, combined, and executed by other modules within the system. It enables consistent, reproducible implementation of standardized task logic across testing, monitoring, and therapeutic contexts while maintaining the flexibility required for individualized adaptation through higher-level system modules.

Further, embodiments may include a patient database 124, which may contain comprehensive patient-specific information used to support assessment, monitoring, and therapy personalization by other system modules. The patient database 124 may store data indicating the stimulation protocols and virtual-reality scenarios presented to each patient, along with the corresponding physiological and behavioral responses recorded by the sensors 136 and microphone 134. Stored information may include performance metrics, health scores, physiological signal recordings used to contextualize functional performance, and metadata such as patient identifiers and time stamps. In some embodiments, the patient database may store patient-specific information indicative of a clinical or functional status of a patient. Such information may include, for example, clinical or diagnostic information, performance- or task-related data, caregiver- or clinician-reported observations, and information indicative of therapy delivery, adherence, or engagement, as well as information derived from sensors or other external systems.

In some embodiments, the patient database 124 may also integrate information originating from external sources, including data from clinical teams, wearable devices, and caregiver or clinician reports submitted through connected applications. Such information may include physiological measurements relevant to symptom context, medication-adherence data, or behavioral observations collected periodically or in response to system prompts. These external inputs may be processed and standardized by the data structuring and exchange module 140 before being written into the patient database 124, ensuring consistency across data types and sources.

In some embodiments, the patient database 124 may be accessible through a user interface of the system and/or remotely by authorized medical teams. Remote access may allow clinicians to review historical and real-time patient data, approve or adjust system-generated therapeutic recommendations, or authorize new treatment protocols based on clinical evaluation. In some embodiments, the system may include one or more software components configured to transmit encrypted subsets of patient data to remote servers or clinical dashboards via the communication interface 106, supporting remote patient monitoring, regulatory compliance, and continuity of care.

Further, embodiments may include a cloud 126, which may be a network of remote servers that provide on-demand computing resources, data storage, or processing services accessible through the Internet. The cloud 126 may consist of one or more distributed servers, storage devices, or networking components that together support remote execution of algorithms, long-term data storage, or high-performance computing tasks.

In some embodiments, the cloud 126 may be used to store or process data transmitted from the Clarity Network 102, such as aggregated patient information, model-training data derived from clinical, symptomatic, or performance-related information, or anonymized usage statistics. In other embodiments, the cloud 126 may support remote-access features for authorized clinicians, such as reviewing patient data or approving updated therapeutic parameters. The cloud 126 may be accessed through various devices—such as computers, tablets, or mobile applications—using secure internet connectivity.

In some embodiments, the cloud 126 may interface with the communication interface 106 to receive or transmit encrypted data, enabling remote patient monitoring, data backup, or integration with external clinical systems when appropriate.

fFurther, embodiments may include a data structuring and exchange module 140, which may be configured to manage both inbound and outbound data flows between the system and external sources. The data structuring and exchange module 140 may include an inbound pipeline that receives data from electronic health record (EHR) systems, wearable devices, or mobile applications through the communication interface 106 or 126. In some embodiments, the module 140 may validate incoming data against consent scopes, normalize formats such as FHIR or HL7, harmonize measurement units, and map medical terminologies (e.g., SNOMED, LOINC, RxNorm) to support interpretation of clinical assessments, symptom reports, and performance-related information. In some embodiments, the data structuring and exchange module 140 may also receive and process information submitted by caregivers or family members through connected applications or external devices linked to the Clarity network 102. Such information may include periodic questionnaires, behavioral observations, medication adherence reports, or contextual data prompted by system-generated notifications. The module 140 may validate and timestamp these entries, structure them into standardized formats, and store them in the patient database 124 for integration with physiological, performance, or environmental data. The processed data may then be written to the patient database 124, where it becomes available for therapeutic decision-making through the scoring module and remote monitoring. The data structuring and exchange module 140 may further include an outbound pipeline that extracts, summarizes, and formats patient data stored in the patient database 124 for clinical review or regulatory reporting. In some embodiments, the module 140 may generate clinician-ready summaries, remote patient monitoring (RPM) dashboards, or standardized documents such as PDF reports or FHIR DiagnosticReports. In some embodiments, data produced by the performance module 110 and stored in the patient database 124 may be structured by the data structuring and exchange module 140 into a report transmitted via the communication interface 106 to authorized medical personnel for review and authorization. In this example, if the clinician approves an updated therapy configuration derived from the scoring and mapping processes, the authorization signal may enable the headset and system 128 to implement the new treatment protocol generated by mapping module 114. If approval is not granted, the system may continue operating under the previously validated protocol. The outbound pipeline may further support data de-identification and export of minimum-necessary information for secure transfer through the communication interface 106 or 126 to external EHR systems or clinician portals. The data structuring and exchange module 140 ensures data integrity, traceability, and compliance across all system communications while maintaining a consistent internal data architecture.

Further, embodiments may include a headset and system 128, which may be a wearable or near-field device configured to deliver immersive virtual environments and multimodal sensory stimulation as defined by the mapping module 114. The headset and system 128 may serve as the primary delivery interface of the Clarity Network 102, providing one or more forms of sensory stimulation—including visual, auditory, vibrotactile, or haptic stimulation—individually or in combination, such as synchronized audiovisual stimulation. In some embodiments, the headset and system 128 may additionally provide monitoring functions that enable data collection before, during, or after therapy sessions to support longitudinal analysis, adaptive control, or safety monitoring.

The headset and system 128 may include high-resolution, high-refresh-rate LCD or OLED displays for rendering virtual environments retrieved from the virtual reality database 118 and modulated according to stimulation parameters defined in the neurostimulation database 120. Speakers 132, bone-conduction transducers, or spatial-audio arrays may provide synchronized auditory stimulation, including rhythmic amplitude modulation, click trains, or tone bursts aligned to therapeutic frequencies. Vibrotactile and haptic actuators may optionally be integrated within the headset or its accessories to deliver tactile entrainment or feedback coordinated with visual and auditory cues.

The headset and system 128 may execute adaptive prompts and interactive sequences as configured by the performance module 110 (for testing or monitoring) or the mapping module 114 (for therapy), using task definitions and prompting configurations retrieved from the task logic database 122. Modules 110 or 114 may determine which hardware components —such as the display, speakers, eye-tracking sensors, microphones, EEG sensors, or haptic actuators—are activated and in what sequence. The headset then carries out the resulting presentation, response capture, and feedback (e.g., gaze-based selection, verbal recall, or motor initiation).

The headset and system 128 may incorporate a multimodal sensing array comprising microphone 134 and sensors 136 for physiological, neural, and behavioral monitoring. These may include EEG electrodes for recording brain activity or, in some embodiments, delivering electrical stimulation (e.g., tACS or tDCS); eye-tracking and pupillometry sensors for gaze and arousal monitoring; microphones for speech analysis; heart-rate and skin-conductance sensors for autonomic assessment; and inertial sensors for motion tracking. In some embodiments, the headset and system 128 may further include one or more cameras—such as inward-facing cameras for facial expression or eye-region monitoring, and outward-facing cameras for environment pass-through, context detection, or tracking upper-limb movement and gesture performance during tasks. Data collected through these sensors may be processed locally by the performance module 110, stored in the memory 116, or transmitted through the communication interface 138 to the patient database 124. When sharing such data with external systems is desired, corresponding records from the patient database 124 may be processed by the data structuring and exchange module 140 before transmission via the communication interface 106.

The headset and system 128 may be ergonomically designed with an adjustable, padded headband for comfortable extended use and may feature a lightweight build to reduce fatigue. In some embodiments, an RGB LED indicator may provide visual feedback on device status, such as activation, pairing mode, or alerts, including medical notifications.

While primarily configured for neuromodulation delivered through embedded audiovisual components, the system may also interface with external neuromodulation devices—such as vagus-nerve stimulators, mechanical stimulators, ultrasound devices, or peripheral electrical stimulators—to support combination therapies defined by the mapping module 114.

The headset and system 128 may communicate with the Clarity Network 102 through wired or wireless connections, supporting both open-loop and closed-loop operation. In some embodiments, onboard processing hardware may execute adaptive logic locally to ensure low-latency responsiveness even without continuous cloud connectivity. Power may be supplied through rechargeable batteries or cradle-based charging, with integrated safety and automatic-shutdown mechanisms intended to prevent overstimulation or data loss.

Through these integrated features, the headset and system 128 function as the delivery and monitoring interface of the Clarity Network 102—executing personalized therapeutic protocols defined by the mapping module 114, combining immersive content from the virtual reality database 118, neuromodulation methods and stimulation parameters from the neurostimulation database 120, and behavioral prompts from the task logic database 122, while continuously or periodically collecting physiological, neural, and behavioral data to support personalized neurostimulation delivery, safety assurance, and remote clinical monitoring.

Further, embodiments may include a screen 130, which may be a high-performance display panel embedded within the headset and responsible for presenting immersive virtual environments and delivering temporally precise visual stimuli for therapeutic interventions. The screen 130 may support dynamic content delivery with high refresh rates, resolution, and brightness, enabling both passive and active engagement with virtual-reality scenarios and sensory-stimulation protocols.

In some embodiments, the screen 130 may be a high-definition or ultra-high-definition LCD or OLED display optimized for therapeutic use. The display may support refresh rates sufficient to accurately present flickering or temporally modulated visual stimuli at target frequencies—such as 40 Hz for gamma-frequency entrainment—while maintaining perceptual stability and minimizing latency. In such embodiments, there may be a requirement that the screen 130 operate at a refresh rate that is an integer multiple of the intended visual-stimulation frequency. For example, where the system provides a 40 Hz visual stimulus, a refresh rate of at least 80 frames per second may be desirable based on Nyquist sampling considerations. Higher-refresh-rate displays (e.g., 120 Hz, 160 Hz, 200 Hz, or 240 Hz) may be used to ensure stable luminance modulation and reduce visual artifacts during immersive rendering.

The screen 130 may further support adjustable brightness, contrast, and color-accuracy settings to enable a wide range of therapeutic scenarios, from calming low-light environments to vivid high-contrast tasks. Stimulation may be delivered through full-environment modulation—where the entire display oscillates at a specified frequency—or through localized luminance modulation applied to objects, textures, or overlays within the virtual scene. In some embodiments, advanced rendering techniques may enable sinusoidal, quasi-sinusoidal, or square-wave luminance modulation to support precise neural entrainment.

The screen 130 may interface with the sensors 136 of the headset and system 128, including eye-tracking and pupillometry sensors, to monitor user engagement and adapt stimuli based on real-time physiological feedback such as gaze behavior, or arousal state measures transmitted through the headset's sensing array. In some embodiments, integrated software may dynamically adjust screen parameters—such as flicker frequency, brightness envelope, contrast modulation depth, color intensity, or spatial patterning—in response to patient-specific requirements or closed-loop therapeutic logic governed by the performance module 110 or mapping module 114.

The screen 130 may also be used to present cognitive, motor, or affective tasks, including gamified tasks, visual-memory tests, or personally meaningful environments constructed from uploaded photographs or familiar scenes, thereby enhancing engagement and supporting individualized therapeutic experiences.

Further, embodiments may include speakers 132, which may be integrated audio-output devices configured to deliver therapeutic auditory stimuli and task-related audio content as part of the sensory-stimulation and virtual-environment experience. In some embodiments, the speakers 132 may be positioned near the user's ears or implemented as bone-conduction transducers, enabling clear delivery of auditory signals while maintaining environmental awareness when appropriate.

In some embodiments, the speakers 132 may be designed to support a wide frequency range with low distortion and high fidelity, ensuring accurate reproduction of modulated auditory patterns used for entrainment—such as amplitude-modulated tones, click trains, or tone bursts aligned to therapeutic frequencies. The system may dynamically synchronize auditory stimuli with visual or tactile stimulation to maintain precise temporal and phase alignment, thereby enhancing multimodal neural entrainment. For example, a 40 Hz auditory modulation may be phase-locked with a 40 Hz visual flicker or combined with complementary-frequency sensory cues to engage targeted neural pathways to amplify the downstream behavioural effects.

In some embodiments, the speakers 132 may provide task-related audio guidance and feedback, including spoken instructions, prompts, questions, or performance-related cues during cognitive, motor, or functional tasks. The speakers 132 may additionally generate immersive soundscapes—such as environmental ambience, music, or personalized audio elements—retrieved from the virtual reality database 118 to increase ecological validity, engagement, and emotional relevance.

In some embodiments, the spatial-audio subsystem may produce three-dimensional auditory fields in which sound direction, distance, and movement are dynamically modulated in synchrony with visual content. Such spatialized auditory cues may support tasks involving spatial navigation, attention guidance, emotional modulation, memory encoding, or motor coordination within the virtual-reality environment.

Further, embodiments may include a microphone 134, which may be an audio-input component integrated within the headset and system 128 and configured to capture user speech, vocalizations, breathing patterns, or other audible signals generated during testing or therapeutic sessions. The microphone 134 may support real-time acquisition of audio data for purposes including task interaction, behavioral assessment, safety monitoring, and communication with external parties.

In some embodiments, the microphone 134 may be used to capture verbal responses during cognitive or functional tasks administered by the performance module 110. Such responses may include spoken answers, word recall, verbal fluency outputs, or vocal commands, which may be analyzed to derive performance-related measures such as response latency, accuracy, speech rate, articulation quality, or hesitation patterns. These measures may be used as indicators of cognitive, linguistic, or affective function relevant to the patient's clinical profile.

In some embodiments, audio signals captured by the microphone 134 may be analyzed to detect behavioral or physiological states associated with discomfort, distress, confusion, agitation, or fatigue. For example, elevated vocal intensity, changes in prosody, repeated verbal expressions of discomfort, labored breathing, coughing, or explicit verbal stop commands may be identified using predefined rules or pattern-recognition models. Upon detection of such signals, the system may initiate safety-related actions, including pausing or terminating stimulation delivery, modifying session parameters, or triggering alerts to caregivers or authorized clinical personnel via the communication interface 106.

In some embodiments, the microphone 134 may enable direct user-initiated control of the system through voice commands, allowing a patient to request assistance, repeat instructions, pause a task, or discontinue a session without requiring physical input. This capability may be particularly relevant for users with limited motor control, visual impairment, or reduced dexterity, supporting accessibility and patient autonomy during unsupervised or home-based use.

In some embodiments, audio data captured by the microphone 134 may be stored in the memory 116 or patient database 124, either in raw or processed form, along with associated metadata such as timestamps, task context, or session identifiers. Such data may be used for longitudinal assessment of speech-related performance, monitoring of symptom progression, or retrospective clinical review, subject to applicable consent and privacy controls.

In some embodiments, the microphone 134 may further support bidirectional communication during remote or semi-supervised therapy, enabling clinicians or caregivers to provide spoken instructions, reassurance, or guidance through the headset and system 128 when appropriate. Audio communication may be initiated manually by authorized parties or automatically by the system in response to detected safety or compliance events.

Through these functionalities, the microphone 134 serves as a primary behavioral and safety-sensing interface within the Clarity Network 102, enabling speech-based task interaction, detection of distress or adverse events, user-initiated interruption of therapy, and communication with caregivers or clinical teams, while supporting safe and adaptive delivery of neuromodulation-based therapeutic sessions.

Further, embodiments may include sensors 136 configured to capture a wide range of physiological and behavioral metrics during testing and therapeutic sessions, providing real-time data that support personalization of therapy delivery, safety monitoring, and interpretation of patient behavior. In some embodiments, the sensors 136 may include eye-tracking sensors for monitoring gaze direction, fixation stability, eyelid position, and eye openness; pupillometry sensors for tracking pupil dilation associated with attention or arousal; and heart-rate or electrodermal sensors for evaluating autonomic activity such as stress, fatigue, or emotional engagement.

In some embodiments, the sensors 136 may further include motion-sensing components such as inertial measurement units (IMUs), accelerometers, and gyroscopes embedded within the headset and system 128 to detect head movement, orientation, tremor, or response initiation latency during task execution. In some embodiments, inward-facing or outward-facing cameras may capture facial expressions, head posture, gross motor behavior, or interaction patterns, enabling assessment of behavioral responsiveness, coordination, or task compliance. Such motion-and vision-based sensing may be particularly useful for evaluating motor function, psychomotor slowing, restlessness, or disengagement during therapeutic or assessment tasks.

In some embodiments, EEG sensors included within sensors 136 may capture neural activity across frequency bands such as theta, alpha, beta, or gamma and may be used as supplementary contextual information to support interpretation of observed behavioral or performance patterns. For example, EEG-derived measures may be considered alongside task outcomes, gaze behavior, or motion data to help distinguish reduced performance due to fatigue, inattention, or transient cognitive fluctuation. EEG signals are not required to directly control therapy delivery but may provide corroborative information relevant to safety monitoring or clinical interpretation.

In some embodiments, multimodal sensor data may be evaluated to detect physiological or behavioral states indicative of increased agitation, distress, or inability to safely continue a session. For example, eye-tracking data may reveal abnormal saccadic patterns, such as reduced saccade frequency, increased saccade latency, or repetitive gaze disengagement from task-relevant stimuli, while pupillometry measurements may indicate atypical pupil dynamics, including sustained constriction or reduced task-evoked pupillary responses, consistent with fatigue. In addition, speech-derived features captured by microphone 134—such as increased vocal strain, elevated speech rate variability, prolonged response latency, repeated verbal interruptions, or changes in prosody associated with agitation or frustration—may provide behavioral indicators of distress or reduced tolerance to the ongoing session.

When such multimodal patterns are detected, either individually or in combination, the system may pause or adapt stimulation, present user-facing prompts or guidance, terminate the session, or optionally notify a caregiver or authorized clinician, thereby supporting user safety and appropriate continuation of therapy.

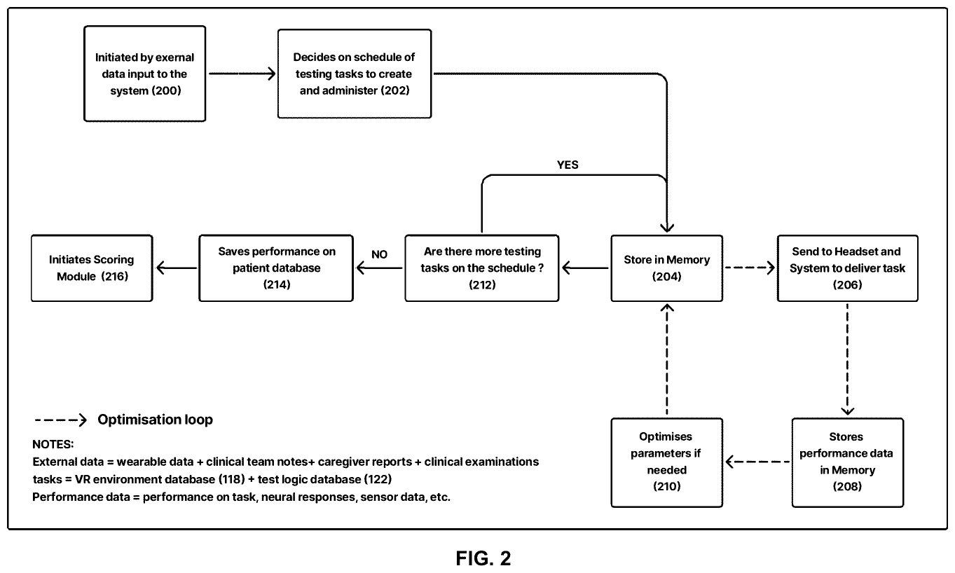

FIG. 2 illustrates an embodiment of the performance module 110, which may be configured to assemble, deliver, and manage testing tasks while collecting associated performance and behavioral data, and other patient-specific information.

The process may begin, at step 200, when the system is initiated based on external information made available to the Clarity Network 102. Such information may include the patient's indication for use, clinical-team inputs, caregiver reports, wearable-device data, or updates received from the data structuring and exchange module 140. In some embodiments, the performance module 110 may also be activated directly by the user through the headset and system 128.

At step 202, the performance module 110 determines a schedule of testing tasks for the session. This schedule may draw from predefined testing protocols associated with the patient's indication or may incorporate external inputs or prior session results. The performance module 110 retrieves the corresponding task definitions from the task logic database 122 and the associated virtual-environment assets from the virtual reality database 118.

At step 204, the performance module 110 stores the generated task schedule and associated configuration parameters in memory 116. These stored entries form the temporary execution plan for the session and allow the system to track task order, difficulty settings, and modality requirements.

At step 206, the performance module 110 retrieves the next task from memory 116 and sends the corresponding environment assets, prompts, and interaction rules to the headset and system 128 for delivery. The headset presents the task to the user through visual, auditory, or multimodal cues, and its sensing array—including eye-tracking sensors, pupillometry sensors, inertial sensors, microphones, inward- or outward-facing cameras, and, in some embodiments, EEG electrodes—collects performance, physiological, and neural data in real time.

At step 208, the performance module 110 receives and stores the collected performance data in memory 116, updating the temporary data record for the ongoing session. Stored data may include behavioral accuracy, response timing, gaze metrics, movement trajectories, speech-based responses, or physiological or task-linked response measures.

At step 210, the performance module 110 may optionally adjust upcoming tasks to maintain clinical relevance or sensitivity to the patient's current functional state. Such optimizations may adjust difficulty, timing, stimulus salience, spatial placement, or other task parameters. Updated task settings may be written back to memory 116 and used when retrieving subsequent tasks in the session.

At step 212, the performance module 110 determines whether additional tasks remain in the stored schedule. If more tasks are present, the module retrieves the next task from memory 116 and returns to step 206 for delivery. If no additional tasks remain, the performance module 110 stores the completed session results in the patient database 124 at step 214, forming part of the patient's longitudinal record.

At step 216, the performance module 110 initiates the scoring module 112, which evaluates the stored results to determine their clinical significance for subsequent mapping and therapeutic adaptation.

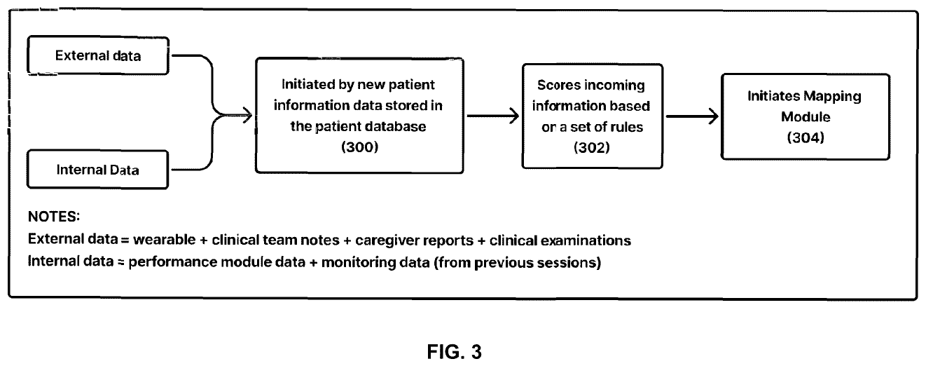

FIG. 3 illustrates an embodiment of the scoring module 112. The process may begin, at step 300, when new patient information becomes available within the patient database 124. This incoming information may originate from external sources—including wearable-device measurements, clinical-team notes, clinician-performed examinations, structured in-clinic assessments, caregiver reports, or medical-record updates received and standardized through the data structuring and exchange module 140—or from internal system sources, such as task-performance outcomes, physiological measurements, neural-activity data, and monitoring results acquired during previous sessions. The scoring module 112 may monitor these updates and initiate its evaluation cycle once new data entries are detected.

At step 302, the scoring module 112 processes and evaluates the newly added information using a set of predefined scoring rules. These rules may incorporate statistical thresholds, rule-based logic, or machine-learning-derived weighting schemes designed to assess the clinical relevance, reliability, and severity of the observed features. The scoring module may analyze integrated data streams that include task accuracy, reaction times, error patterns, movement trajectories, neural-oscillation signatures derived from EEG, gaze patterns and pupillary responses extracted from eye-tracking signals, speech-analysis features obtained from microphone recordings, and autonomic indicators such as heart-rate variability or electrodermal activity. Clinical information—such as findings from neurological examinations, clinician-documented observations, or updates in medical notes—may also be interpreted within the same scoring framework. During this process, the scoring module may perform data-quality validation, such as identifying corrupted eye-tracking segments, inconsistent signals across sensors, or incomplete entries, ensuring that only reliable and interpretable information contributes to the scoring results.

The scoring module 112 may compute domain-specific and composite scores summarizing cognitive, motor, affective, or autonomic, producing an interpretable representation of the patient's current functional status. These scores may be contextualized relative to the patient's historical data, expected therapeutic trajectory, or relevant clinical benchmarks stored within the system.

At step 304, the scoring module 112 initiates the mapping module 114, which may subsequently use these updated scoring outputs to determine whether the therapeutic protocol —including VR environments, neurostimulation methods and parameters, task schedules, or session-level configurations—should be adjusted for the treatment session that is to follow.

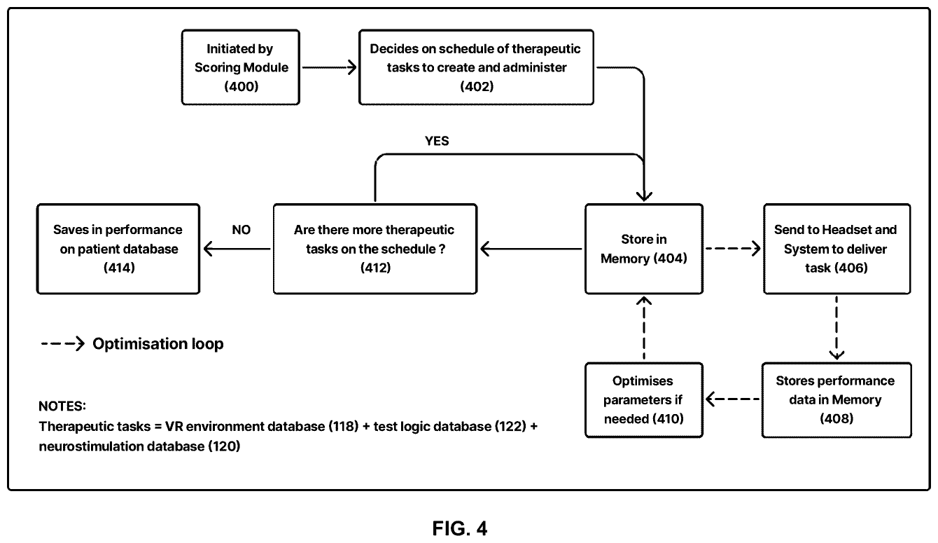

FIG. 4 illustrates the mapping module 114, which may be configured to generate, adjust, and schedule individualized therapeutic tasks based on the significance-weighted results produced by the scoring module 112. The process begins at step 400, when the mapping module 114 is initiated following completion of the scoring cycle. The module retrieves the newly computed scores, physiological summaries, and contextual information from the patient database 124. At step 402, the mapping module 114 determines a schedule of treatment tasks to create and administer. This treatment schedule may include one or more task components derived from the neurostimulation database 120, the virtual reality database 118, and the task logic database 122, and may prioritize the domains showing the greatest impairment according to the scoring outcome, caregiver observations, or longitudinal progression. At step 404, the selected therapeutic configuration is stored temporarily in memory 116, including task ordering, stimulation parameters, associated VR assets, difficulty settings, and any adaptive rules. At step 406, the mapping module 114 transmits the next treatment task to the headset and system 128 for delivery, enabling presentation of the corresponding sensory stimulation, VR environment elements, and task-logic instructions. As the patient performs the therapeutic activity, physiological and behavioral information is collected by the sensors 136 and microphone 134 and is stored in memory at step 408. At step 410, the mapping module 114 may optionally adjust stimulation parameters, task difficulty, or session duration based on updated scoring outputs or session-level performance indicators indicative of therapeutic relevance or effectiveness. In some embodiments, such adjustments may be applied during a treatment session, between sessions, or across successive treatment cycles as part of a longitudinal personalization strategy.

If adaptation is applied, the updated task configuration is saved in memory 116. At step 412, the system determines whether additional therapeutic tasks remain in the schedule. If tasks remain, the process returns to step 404 to retrieve and deliver the next intervention. If no additional tasks remain, the mapping module 114 stores the session summary in the patient database 124 at step 414 and completes the cycle.

In one example, the system applies a baseline therapeutic protocol for mild Alzheimer's disease that delivers 40 Hz flicker stimulation embedded within a set of cognitive tasks, including word recall, visual discrimination, face-name association, and spatial-navigation activities such as path finding. These same domains are assessed through the performance module 110. After reviewing the scored results, the scoring module 112 identifies a consistent pattern of poorer performance in spatial-navigation tasks, compared to performance in the other domains. Caregiver reports of increasing difficulty with wayfinding further reinforce this finding. Based on this convergence of evidence, the mapping module 114 adjusts the patient's personalized protocol by increasing the spatial-navigation component—for example, by raising cognitive load, extending task duration, or increasing weekly frequency—while maintaining the baseline structure of the other therapeutic tasks. Over multiple weeks of therapy, a patient shows sustained improvement in visuospatial tasks that were initially prioritized. However, performance in word-finding and semantic-retrieval tasks—such as category fluency, picture naming, and semantic-association matching—begins to plateau or decline. Caregiver reports noting increased difficulty finding words in conversations further reinforce this trend.

The scoring module 112 increases the weighting of the language/semantic-memory domain, and the mapping module 114 adjusts the therapeutic protocol accordingly—for example, by increasing the frequency or duration of semantic-retrieval exercises embedded within the 40 Hz stimulation framework, while reducing emphasis on visuospatial tasks that have already improved. This ensures treatment focus evolves with the patient's changing clinical profile.

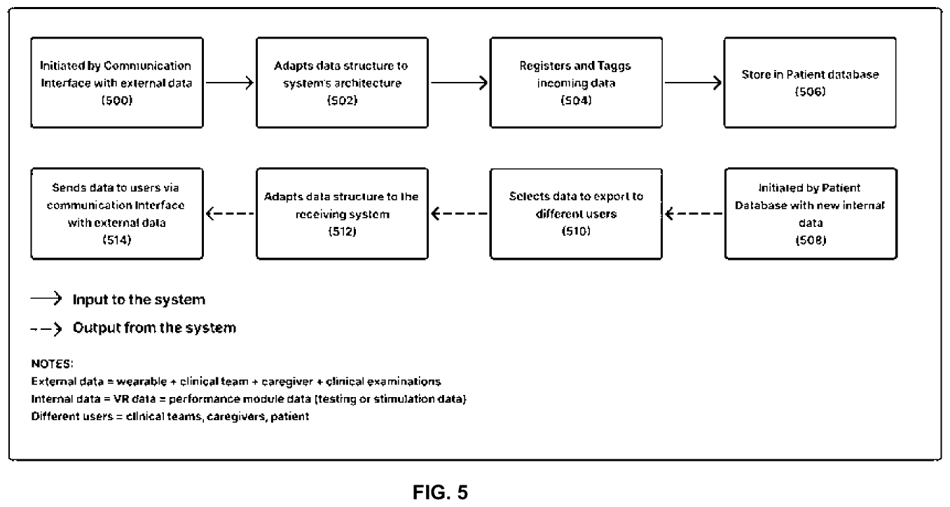

FIG. 5 illustrates the data structuring and exchange module 140, which may be configured to manage inbound and outbound data flows between the Clarity Network 102 and external systems. The process begins at step 500, when the module is initiated by the receipt of external data through the communication interface 106 or 126. External inputs may include wearable-device measurements, clinical-team notes, caregiver reports, medical examinations, or other health-information sources. At step 502, the module adapts these incoming data to the system's internal architecture by validating formats, normalizing measurement units, aligning data elements with internal schema, and mapping clinical terminologies to standardized vocabularies when appropriate. At step 504, the data structuring and exchange module 140 processes external information received through the communication interface by registering and categorizing the incoming data according to predefined data types supported by the system. External information may include, for example, clinical assessments, physiological measurements, caregiver-provided observations, wearable-device outputs, imaging summaries, laboratory values, or other health-related information, without performing therapeutic interpretation or clinical evaluation at this stage. At step 506, the registered external information is stored in the patient database 124 in association with the corresponding patient record, where it becomes available to other system modules, including the performance module 110 and the scoring module 112, subject to system configuration and access controls. At step 508, the process may alternatively be initiated by internally generated data, such as task-performance outcomes, behavioral responses, or physiological signals recorded during testing or treatment sessions and written to the patient database 124. At step 510, the data structuring and exchange module 140 selects portions of the internal dataset for outbound communication based on predefined data-sharing configurations associated with different external recipients. The selection may reflect reporting preferences, access permissions, or communication settings, without determining clinical relevance or therapeutic significance. At step 512, the module restructures the selected internal information into formats compatible with the receiving system, such as converting to standardized clinical documents, generating clinician-ready summaries, or producing structured data packages aligned with interoperability or reporting requirements. At step 514, the module transmits the adapted data through the communication interface 106 or 126 to the appropriate external destination, such as clinical dashboards, caregiver applications, or patient-facing portals. Through these functions, the data structuring and exchange module 140 ensures consistent data integrity, bidirectional interoperability, and continuity between internal therapeutic logic and external clinical ecosystems, while preserving traceability and compliance across all data exchanges.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

What is claimed is:

1. A method for personalized neuromodulation therapy delivered within virtual reality environments, the method comprising:
storing in memory, a plurality of therapeutic tasks associated with one or more rules and one or more virtual environment assets, wherein the therapeutic tasks target different functional domains;
providing, via a headset system, a therapeutic protocol that includes a combination of modes of stimulation and one or more sets of the therapeutic tasks;
scoring sensor data received from the headset system in response to the therapeutic protocol and external clinical data, wherein the scoring is based on one or more metrics; and
adjusting the therapeutic protocol based on the scores, wherein adjusting the therapeutic protocol includes modifying one or more parameters of the therapeutic protocol based on relative performance of different tasks.

2. The method of claim 1, wherein the functional domains include cognitive, behavioral, or motor functions.

3. The method of claim 1, wherein adjusting the therapeutic protocol further includes changing a type, order, or duration of the therapeutic tasks.

4. The method of claim 1, wherein adjusting the therapeutic protocol further includes changing a difficulty of the therapeutic tasks.

5. The method of claim 1, wherein adjusting the therapeutic protocol further includes changing a spatial placement of the virtual environment assets.

6. The method of claim 1, wherein adjusting the therapeutic protocol further includes changing one or more parameters of the neuromodulation stimulation.

7. The method of claim 1, further comprising receiving, via a communication interface, the external clinical data from one or more third party systems.

8. The method of claim 1, wherein the scores are weighted based on relevance to one or more functional deficits of a user.

9. The method of claim 1, wherein the scores are weighted based on a severity of one or more functional deficits of a user.

10. The method of claim 1, wherein at least one of the modes of stimulation includes sensory stimuli provided at gamma frequencies.

11. A system for personalized neuromodulation therapy delivered within virtual reality environments, the system comprising:
memory that stores a plurality of therapeutic tasks associated with one or more rules and one or more virtual environment assets, wherein the therapeutic tasks target different functional domains; and
a processor that executes instructions in memory, wherein the processor executes instructions to:
provide, via a headset system, a therapeutic protocol that includes a combination of modes of stimulation and one or more sets of the therapeutic tasks;
score sensor data received from the headset system in response to the therapeutic protocol and external clinical data, wherein the scoring is based on one or more metrics; and
adjust the therapeutic protocol based on the scores, wherein adjusting the therapeutic protocol includes modifying one or more parameters of the therapeutic protocol based on relative performance of different tasks.

12. The system of claim 11, wherein the functional domains include cognitive, behavioral, or motor functions.

13. The system of claim 11, wherein the therapeutic protocol is further adjusted by changing a type, order, or duration of the therapeutic tasks.

14. The system of claim 11, wherein the therapeutic protocol is further adjusted by changing a difficulty of the therapeutic tasks.

15. The system of claim 11, wherein the therapeutic protocol is further adjusted by changing a spatial placement of the virtual environment assets.

16. The system of claim 11, wherein the therapeutic protocol is further adjusted by changing one or more parameters of the neuromodulation stimulation.

17. The system of claim 11, further comprising a communication interface that receives an external clinical information from one or more third party systems.

18. The system of claim 11, wherein the scores are weighted based on relevance to one or more functional deficits of a user.

19. The system of claim 11, wherein at least one of the modes of stimulation includes sensory stimuli provided at gamma frequencies.

20. A non-transitory, computer-readable storage medium, having embodied thereon a program executable by a processor to perform a method for personalized neuromodulation therapy delivered within virtual reality environments, the method comprising:
storing in memory, a plurality of therapeutic tasks associated with one or more rules and one or more virtual environment assets, wherein the therapeutic tasks target different functional domains;
providing, via a headset system, a therapeutic protocol that includes a combination of modes of stimulation and one or more sets of the therapeutic tasks;
scoring sensor data received from the headset system in response to the therapeutic protocol and external clinical data, wherein the scoring is based on one or more metrics; and
adjusting the therapeutic protocol based on the scores, wherein adjusting the therapeutic protocol includes modifying one or more parameters of the therapeutic protocol based on relative performance of different tasks.
